(19) United States
(12) Patent Application Publication
(10) Pub. No.: US 2026/0031235 A1
(43) Pub. Date: Jan. 29, 2026

(54) VITAL DATA EVALUATION METHOD, VITAL DATA EVALUATION DEVICE, VITAL DATA EVALUATION SYSTEM, AND NON-TRANSITORY COMPUTER-READABLE MEDIUM

(71) Applicant: SHIMADA PATENTS CORPORATION, Ibaraki (JP)

(72) Inventors: Kensuke SHIMADA, Ibaraki (JP); Masahiko GOSHO, Chiba (JP); Ryota ISHII, Chiba (JP)

(21) Appl. No.: 19/344,358

(22) Filed: Sep. 29, 2025

Related U.S. Application Data
(63) Continuation of application No. PCT/JP2025/019403, filed on May 29, 2025.

(30) Foreign Application Priority Data
Jun. 12, 2024 (JP) 2024-095300

(51) Int. Cl. G16H 50/30; G16H 10/60

(52) U.S. Cl. CPC G16H 50/30; G16H 10/60

(57) ABSTRACT

The vital data evaluation method comprises acquiring a first vital data set corresponding to a first type of vital data of a patient; acquiring a second vital data set corresponding to a second type of vital data of the patient that is different from the first type of vital data; generating first vital data function information based on the first vital data set; generating second vital data function information based on the second vital data set; and generating patient status information for estimating a status of the patient based on a relationship between the first vital data function information and the second vital data function information.

### CROSS-REFERENCE TO RELATED APPLICATIONS

This application is a Continuation of International Patent Application No. PCT/JP2025/19403, filed on May 29, 2025, which claims the benefit of priority to Japanese Patent Application No. 2024-095300, filed on Jun. 12, 2024, the entire contents of which are incorporated herein by reference.

### FIELD

The present disclosure relates to a vital data evaluation method, a vital data evaluation device, a vital data evaluation system, and a non-transitory computer-readable medium.

### BACKGROUND

Vital (sign) data, which is a data compilation of “vital signs” indicating that a person is alive, is widely used in medical settings and daily health management. For example, in medical settings, each data, such as pulse rate, blood pressure, body temperature, and respiratory rate, is recorded as vital data.

Japanese laid-open patent publication No. 2013-220175 discloses an embodiment in which a heart rate is calculated by acquiring and processing electrocardiogram data and pulse wave data.

### SUMMARY

According to one embodiment of the present invention, a vital data evaluation method is provided comprising: acquiring a first vital data set corresponding to a first type of vital data of a patient; acquiring a second vital data set corresponding to a second type of vital data different from the first type of vital data of the patient; generating first vital data function information based on the first vital data set; generating second vital data function information based on the second vital data set; and generating patient status information for estimating a patient's status based on a relationship between the first vital data function information and the second vital data function information.

The above vital data evaluation method may further comprise excluding first vital data that exceeds a preset numerical range from the first vital data set; excluding second vital data that exceeds a preset numerical range from the second vital data set; generating the first vital data function information based on the first vital data set from which the first vital data exceeding the preset numerical range has been excluded; and generating the second vital data function information based on the second vital data set from which the second vital data exceeding the preset numerical range has been excluded.

The above vital data evaluation method may further comprise generating first vital data derivative function information based on the first vital data function information; generating second vital data derivative function information based on the second vital data function information; and generating patient status information based on a patient status data set corresponding to the first vital data derivative function information, the second vital data derivative function information, and a relationship therebetween.

In the above vital data evaluation method, the first and second vital data derivative function information are generated when at least one of the first and second vital data function information satisfies a predetermined condition.

The above vital data evaluation method may further comprise generating action information for treating or managing the patient, based on an action information data set associated with the patient status information.

The above vital data evaluation method may further comprise generating severity information based on at least one of the first and second vital data function information; and generating the action information based on the severity information and the patient status information.

The above vital data evaluation method may further comprise generating warning information when at least one of the first and second vital data function information satisfies a predetermined condition.

The above vital data evaluation method may further comprise outputting first identification information associated with the first vital data derivative function information and second identification information associated with the second vital data derivative function information.

In the above vital data evaluation method, the first identification information is information that visually represents a transition of the first vital data; and the second identification information is information that visually represents a transition of the second vital data.

The above vital data evaluation method may further comprise selecting a basis function to be applied from a plurality of basis functions, based on at least one type of vital data, information about the patient, and external information associated with the patient, when generating at least one of the first and second vital data function information.

The above vital data evaluation method may further comprise generating the patient status information by applying the first and second vital data function information to a pre-generated machine learning model.

In the above vital data evaluation method, the machine learning model corresponds to shape patterns of the first and second vital data function information.

According to one embodiment of the present invention, a non-transitory computer-readable medium is provided storing a program that causes a computer to execute the above vital data evaluation method.

According to one embodiment of the present invention, a vital data evaluation device is provided comprising: a processor; and a memory device configured to store a program that causes the processor to: acquire a first vital data set corresponding to a first type of vital data of a patient; acquire a second vital data set corresponding to a second type of vital data different from the first type of vital data of the patient; generate first vital data function information based on the first vital data set; generate second vital data function information based on the second vital data set; and generate patient status information for estimating a patient's status based on a relationship between the first vital data function information and the second vital data function information.

In the above vital data evaluation device, the program may cause the processor to: exclude first vital data that exceeds a preset numerical range from the first vital data set; exclude second vital data that exceeds a preset numerical range from the second vital data set; generate the first vital data function information based on the first vital data set from which the first vital data exceeding the preset numerical range has been excluded; and generate the second vital data function information based on the second vital data set from which the second vital data exceeding the preset numerical range has been excluded.

In the above vital data evaluation device, the program may cause the processor to: generate first vital data derivative function information based on the first vital data function information; generate second vital data derivative function information based on the second vital data function information; and generate the patient status information based on a patient status data set corresponding to the first vital data derivative function information, the second vital data derivative function information, and a relationship therebetween.

In the above vital data evaluation device, the program may cause the processor to generate the first and second vital data derivative function information when at least one of the first and second vital data function information satisfies a predetermined condition.

In the above vital data evaluation device, the program may cause the processor to generate action information for treating or managing the patient, based on an action information data set associated with the patient status information.

In the above vital data evaluation device, the program may cause the processor to generate severity information based on at least one of the first and second vital data function information; and generate the action information based on the severity information and the patient status information.

In the above vital data evaluation device, the program may cause the processor to generate warning information when at least one of the first and second vital data function information satisfies a predetermined condition.

In the above vital data evaluation device, the program may cause the processor to output first identification information associated with the first vital data derivative function information and second identification information associated with the second vital data derivative function information.

In the above vital data evaluation device, the first identification information is information that visually represents a transition of the first vital data; and the second identification information is information that visually represents a transition of the second vital data.

In the above vital data evaluation device, the program may cause the processor to select a basis function to be applied from a plurality of basis functions, based on at least one type of vital data, information about the patient, and external information associated with the patient, when generating at least one of the first and second vital data function information.

In the above vital data evaluation device, the program may cause the processor to generate the patient status information by applying the first and second vital data function information to a pre-generated machine learning model.

In the above vital data evaluation device, the machine learning model corresponds to shape patterns of the first and second vital data function information.

According to one embodiment of the present invention, a vital data evaluation system is provided comprising: a processor; and a memory device configured to store a program that causes the processor to: acquire a first vital data set corresponding to a first type of vital data of a patient; acquire a second vital data set corresponding to a second type of vital data different from the first type of vital data of the patient; generate first vital data function information based on the first vital data set; generate second vital data function information based on the second vital data set; and generate patient status information for estimating a patient's status based on a relationship between the first vital data function information and the second vital data function information.

In the above vital data evaluation device, the program may cause the processor to exclude first vital data that exceeds a preset numerical range from the first vital data set; exclude second vital data that exceeds a preset numerical range from the second vital data set; generate the first vital data function information based on the first vital data set from which the first vital data exceeding the preset numerical range has been excluded; and generate the second vital data function information based on the second vital data set from which the second vital data exceeding the preset numerical range has been excluded.

According to the present disclosure, it is possible to easily determine a patient's status similar to that of a skilled medical professional.

### BRIEF DESCRIPTION OF DRAWINGS

FIG. 1 is a diagram illustrating a vital data evaluation system according to an embodiment of the present disclosure.

FIG. 2 is a diagram illustrating a hardware configuration of each device forming a vital data evaluation system according to an embodiment of the present disclosure.

FIG. 3 is a diagram illustrating a software configuration of a control unit of a vital data evaluation server according to an embodiment of the present disclosure.

FIG. 4 is a diagram showing a basis function data table according to an embodiment of the present disclosure.

FIG. 5 is a diagram showing a vital sign-basis function data table according to an embodiment of the present disclosure.

FIG. 6 is a diagram showing a patient status information data table according to an embodiment of the present disclosure.

FIG. 7 is a diagram showing an action information data table according to an embodiment of the present disclosure.

FIG. 8 is a flowchart showing a vital data evaluation process of a vital data evaluation system according to an embodiment of the present disclosure.

FIG. 9 is a flowchart showing a vital data evaluation process of a vital data evaluation system according to an embodiment of the present disclosure.

FIG. 10 is a flowchart showing a vital data evaluation process of a vital data evaluation system according to an embodiment of the present disclosure.

FIG. 11 is a flowchart showing a vital data evaluation process of a vital data evaluation system according to an embodiment of the present disclosure.

FIG. 12 is a flowchart showing a vital data evaluation process of a vital data evaluation system according to an embodiment of the present disclosure.

FIG. 13 is an example of acquired first vital data according to an embodiment of the present disclosure.

FIG. 14 is an example showing a fitting state of a function model according to an embodiment of the present disclosure.

FIG. 15 is an example of a GCV graph according to an embodiment of the present disclosure.

FIG. 16 is an example of vital data function information and vital data derivative function information.

FIG. 17 is a flowchart showing a vital data evaluation process of a vital data evaluation system according to an embodiment of the present disclosure.

FIG. 18 is a flowchart showing a vital data evaluation process of a vital data evaluation system according to an embodiment of the present disclosure.

FIG. 19 is a diagram showing an action information data table according to an embodiment of the present disclosure.

FIG. 20 is a flowchart showing a vital data evaluation process of a vital data evaluation system according to an embodiment of the present disclosure.

FIG. 21 is a transition marker table of vital data function information according to an embodiment of the present disclosure.

FIG. 22 is a flowchart showing a vital data evaluation process of a vital data evaluation system according to an embodiment of the present disclosure.

FIG. 23 is a flowchart showing a method for generating a machine learning model according to an embodiment of the present disclosure.

FIG. 24 is an example of a function shape pattern according to an embodiment of the present disclosure.

### DESCRIPTION OF EMBODIMENTS

The following embodiments are examples of embodiments of the present disclosure, and the present disclosure is not limited to these embodiments. In the drawings referred to in the present embodiment, the same or similar parts are denoted by the same reference signs or similar reference signs, and repeated description thereof may be omitted.

In medical settings, vital data may be measured over time. In this case, medical professionals assess a patient's status based on the transition and trends in multiple vital data, and provide appropriate medical care. However, it is difficult for medical professionals to constantly monitor the same subject and keep track of the patient's status.

The present disclosure discloses facilitating the determination of a patient's status similar to that of a skilled medical professional.

### First Embodiment

A vital data evaluation system according to the present embodiment will be described with reference to the drawings.

### [1-1. Overall Configuration of Vital Data Evaluation System]

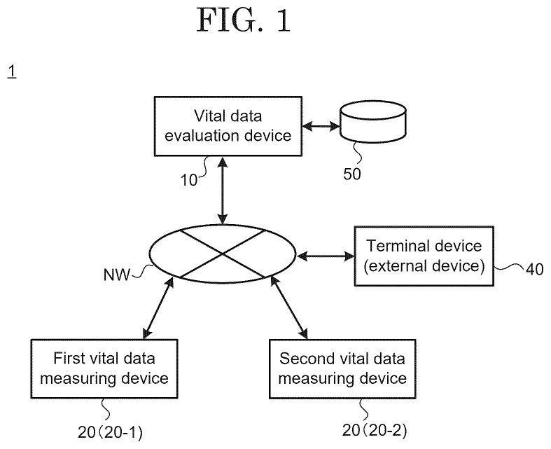

FIG. 1 is a diagram illustrating a vital data evaluation system 1 according to the present embodiment. The vital data evaluation system 1 includes a vital data evaluation device 10, a first vital data measuring device 20-1, a second vital data measuring device 20-2, and a terminal device (external device) 40. In addition, when it is not necessary to distinguish between the first vital data measuring device 20-1 and the second vital data measuring device 20-2, they will be described as the vital data measuring device 20. Further, although one of each of the first vital data measuring device 20-1 and the second vital data measuring device 20-2 is provided in this example, the present disclosure is not limited to this. As described below, a plurality of vital data measuring devices may be used when measuring one type of vital data.

A database 50 is connected to the vital data evaluation device 10. In FIG. 1, the vital data evaluation device 10, the first vital data measuring device 20-1, the second vital data measuring device 20-2, and the terminal device 40 are connected to a network NW. The network NW is a communication network such as the Internet or an intranet, and an appropriate network is used depending on the communication environment.

According to the vital data evaluation system 1, two types of vital data can be measured using the two types of vital data measuring devices 20, and a function of each vital data for a certain time (vital data function information) can be generated. In the present embodiment, systolic blood pressure data and heart rate data measured during surgery under general anesthesia are used as the two types of vital data.

Further, the vital data in an embodiment of the present disclosure includes not only basic information such as heart rate, pulse rate, blood pressure, pulse pressure, body temperature, respiration, and oxygen saturation, but also detailed biometric information obtained from an electrocardiogram, pacemaker, respiratory apparatus, arterial blood pressure measuring device, wearable device, etc. (arrhythmia, electric resistance, heart sound, fluctuation of the electrocardiogram, respiratory changes in blood pressure and pulse wave, pressure measurement value of each site [venous pressure, central venous pressure, pulmonary artery pressure, pulmonary artery wedge pressure, pulmonary vein pressure, atrial pressure, ventricular pressure, arterial pressure, etc.], blood flow speed, respiratory sound, breathing pattern, rhythm of respiration, apnea, ventilation volume, expiratory and inspiratory flow rates, airway pressure, gas analysis [carbon dioxide partial pressure, oxygen partial pressure, inhaled anesthetic concentration, etc.], cardiac output, shock index, amount of exercise, type of exercise, tissue pressure, etc.), state of consciousness, cognitive function, electroencephalogram, sleep, response to external stimuli, intracranial pressure, cerebral blood flow, pupil diameter, pain, itching, nausea, vomiting, speech, test values (blood glucose level, blood cells, hemoglobin level, inflammation, leakage enzymes, renal function, hormones, electrolytes, etc.), muscle tone, muscle relaxation, peripheral coldness, cyanosis, skin condition, body weight, food intake, water intake, urine volume, stool volume, bleeding volume, fetal heart rate, fetal growth and condition, amniotic fluid properties, uterine contractions, degree of cervical opening, and the like. The vital data also includes information based on biological electrical activity, physical, chemical, optical, and acoustical changes, and multimodal information that combines them. In addition, the vital data is not limited to the format of consecutive values, discrete values, images, waveforms, sounds, videos, and the like, and may also include features extracted from these, and indicators generated by AI (Artificial Intelligence). Further, the vital data in the present disclosure is not limited to biometric information generally used at present, and is interpreted to include any information that may be recognized as an indicator of physiological or pathological conditions in the medical field or research field in the future.

In addition, the vital data evaluation device 10 generates patient status information for estimating the patient's status based on the relationship between the two types of vital data function information, and further generates action information for treating the patient. The generated functional information, patient status information, and action information are displayed on the vital data evaluation device 10 (or the terminal device 40). Hereinafter, a configuration of the vital data evaluation system 1 for realizing such a process will be described.

### [1-2. Hardware Configuration]

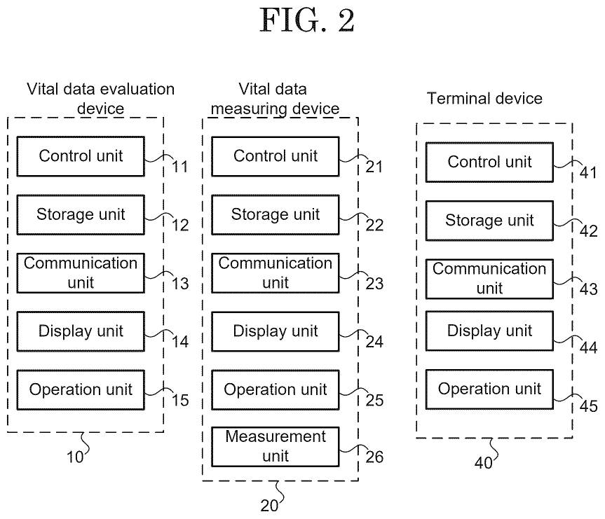

FIG. 2 is a diagram illustrating a hardware configuration of each device forming the vital data evaluation system 1.

### [1-2-1. Vital Data Evaluation Device 10]

The vital data evaluation device 10 includes a control unit 11, a storage unit 12 and a communication unit 13, a display unit 14, and an operation unit 15. The vital data evaluation device 10 may be an on-premises server, a cloud server, or another processing device. The control unit 11 is an example of a computer including an arithmetic processing circuit, such as a CPU (Central Processing Unit), an ASIC (Application Specific Integrated Circuit), and an FPGA (Field Programmable Gate Array). The control unit 11 executes programs stored in the storage unit 12 to realize various functions in the vital data evaluation device 10. As a result, the vital data evaluation device 10 executes a process (vital data evaluation process) in the vital data evaluation system 1.

The storage unit 12 includes a storage device and stores a control program. In addition, the storage unit 12 also stores various types of data used when the control program is executed. Further, the program may be provided in a state of being recorded on a computer-readable recording medium, such as a magnetic recording medium, an optical recording medium, a magneto-optical recording medium, or a semiconductor memory.

In this case, the vital data evaluation device 10 may include an interface for connecting the recording medium. In this case, the recording medium may be defined as a medium different from the storage unit 12 included in the vital data evaluation device 10, or may be a medium used in the storage unit 12.

The communication unit 13 includes a communication module, and is connected to the network NW under the control of the control unit 11 to transmit and receive data to and from other devices connected to the network NW. In this example, the communication unit 13 is also connected to the data base 50 to transmit and receive information. Information registered in the database 50 will be described later. The communication unit 13 may be connected to the database 50 via the network NW. In addition, the information registered in the database 50 may be stored in the storage unit 12. In this case, the database 50 may not be present.

The display unit 14 includes a display device whose content is controlled by the control unit 11. The operation unit 15 may include a keyboard, a switch, a handle, and the like, and outputs information corresponding to the operation to a control unit 21.

Further, in addition to the above-described configuration, the vital data evaluation device 10 may have other configurations, such as an audio output unit and a light-emitting unit.

[1-2-2. Vital Data Measuring device 20]

The vital data measuring device 20 includes the control unit 21, a storage unit 22, a communication unit 23, a display unit 24, an operation unit 25, and a measurement unit 26. The control unit 21 has a configuration basically similar to that of the control unit 11 described above, and realizes various functions in the vital data measuring device 20. As a result, processing in the vital data measuring device 20 in the vital data evaluation system 1 is executed. The measured vital data may be stored in the storage unit 22 via the control unit 21.

The storage unit 22 has a configuration basically similar to that of the storage unit 12, and only the contents of the instructions of the stored program are different, so that the description thereof will be omitted. The communication unit 23 has basically the same configuration as that of the communication unit 13, and since the connectable networks differ from each other, the explanation thereof will be omitted.

The display unit 24 has a configuration basically similar to that of the display unit 14, and can display the acquired vital data. The operation unit 25 has a configuration similar to that of the operation unit 15, and only the contents to be operated are different, so that the description thereof will be omitted.

The measurement unit 26 is used to measure vital data of a patient.

The measurement unit 26 may invasively measure vital data or may non-invasively measure vital data.

### [1-2-3. Terminal Device 40]

The terminal device 40 is composed of at least one of a desktop-type PC (personal computer), a notebook-type PC, a mobile telephone, a smart phone, a tablet-type terminal, and another electronically-applied mechanical device. The terminal device 40 can function as a client. The terminal device includes a control unit 41, a storage unit 42, a communication unit 43, a display unit 44, and an operation unit 45. The control unit 41 has basically the same configuration as the control unit 11 described above, and realizes various functions in the terminal device 40. The process in the terminal device 40 in the vital data evaluating system 1 is executed.

The storage unit 42 has a configuration basically similar to that of the storage unit 12, and only the contents of the instructions of the stored program are different, so that the description thereof will be omitted. The communication unit 43 has a configuration basically similar to that of the communication unit 13, and only the connectable networks are different, so that the description thereof will be omitted.

The display unit 44 includes a display device whose content is controlled by the control unit 41. In this example, the operation unit 45 includes a touch sensor, and outputs information corresponding to a position operated by a user (for example, a medical professional) to the control unit 41. The touch sensor is provided on a display region of the display unit 44. That is, the display unit 44 and the operation unit 45 form a touch panel. In addition, the touch panel may also be used in the vital data evaluation device 10 and the vital data measuring device 20.

### [1-3. Software Configuration of Vital Data Evaluation System]

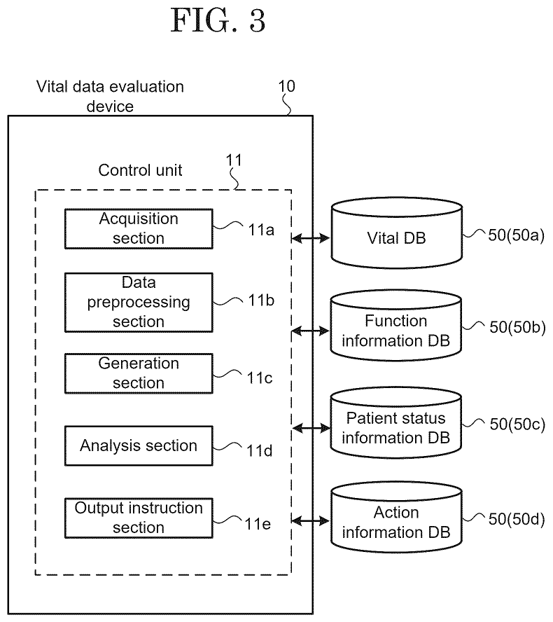

FIG. 3 is a diagram showing a software configuration of the control unit 11 of the vital data evaluation device 10. The control unit 11 includes an acquisition section 11a, a data pre-processing section 11b, a generation section 11c, an analysis section 11d, and an output instruction section 11e.

The acquisition section 11a has a function of acquiring various types of information from each device. In this example, vital data is stored in a vital database (DB) 50a of the database 50. The function information is stored in a function information database (DB) 50b. The patient status information is stored in a patient status information database (DB) 50c.The action information is stored in an action information database (DB) 50d.

The data pre-processing section 11b has a function of excluding (pre-processing, cleaning) data conforming to a predetermined condition from the acquired log data. In this example, the data pre-processing section 11b excludes data that exceeds a preset range.

The analysis section 11d has a function of analyzing a patient's status using the generated vital data function information.

The generation section 11c has a function of generating various types of information. In the present embodiment, the function information is generated using the acquired vital data. In this example, a fitting curve is generated by a function model that best fits a vital data set in which vital data for a certain period is arranged in time series. In addition, the generation section 11c generates patient status information based on the relationship between the two types of vital data functional information. Further, the generation section 11c generates action information for proposing an action based on the patient status information.

The output instruction section 11e has a function of instructing the output of the patient status information and the action information. In this example, the output instruction section instructs the display unit 14 to display the patient status information and the action information, or instructs the terminal device 40 to output the patient status information and the action information.

### [1-4. Various Tables]

Next, various data tables used in the vital data evaluation system 1 will be described.

### [1-4-1. Basis Function Data Table]

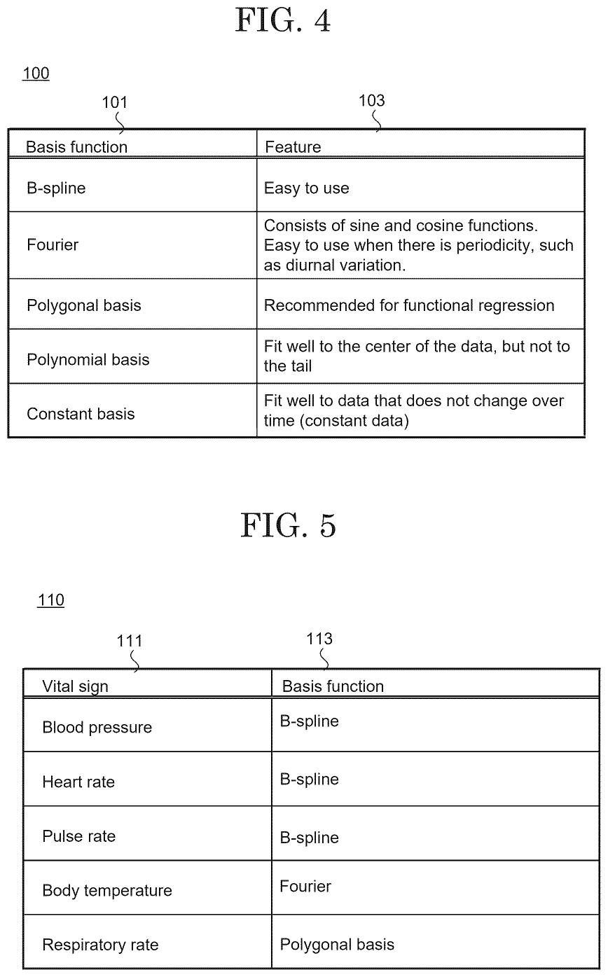

FIG. 4 is a basis function data table 100. The basis function data table 100 and a vital sign-basis function data table 110 are stored in the function data base (DB) 50b. The basis function data table 100 includes basis function (type name) information 101 and feature information 103. The basis function data table 100 includes a basis function prepared in advance. In the basis function data table 100, basis functions can be added or modified as appropriate.

### [1-4-2. Vital Sign-Basis Function Data Table]

FIG. 5 is the vital sign-basis function data table 110. The vital sign-basis function data table 110 is stored in the function data base (DB) 50b. The vital sign-basis function data table 110 includes vital sign (type name) information 111 and basis function (type name) information 113. In the present embodiment, a basis function to be used may be associated with each vital sign. In this example, in the case where the vital sign represents “systolic blood pressure,” the B-spline is associated as a basis function (model) for generating the function information. In addition, the vital sign-basis function data table 110 can be updated as appropriate. Further, in the case of a function model that does not require a basis function, selection of the basis function is not necessary.

### [1-4-3. Patient Status Data Table]

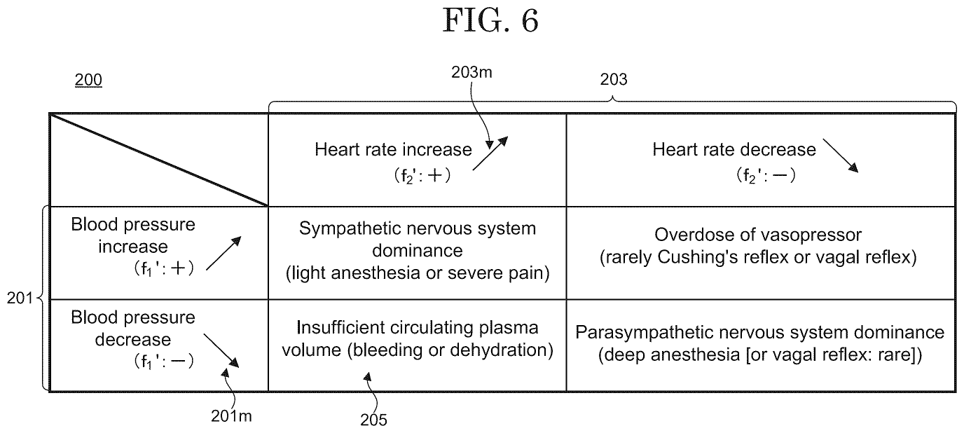

FIG. 6 is a patient status data table 200. The patient status data table 200 includes first vital data derivative function information (f₁′) 201, first identification information 201m, second vital data derivative function information (f₂′) 203, second identification information 203m, and patient status information 205. In the patient status data table 200, patient status information based on the relationship between the first vital data derivative function information f₁′ generated from the first vital data function information and the second vital data derivative function information f₂′ generated from the second vital data function information is shown. For example, in the case where the first vital data function information f₁′ is negative (−) (decrease in systolic blood pressure) and the second vital data function information f₂′ is positive (+) (increase in heart rate), the patient status information “insufficient circulating plasma volume” is associated.

[1-4-4. Action information Data Table]

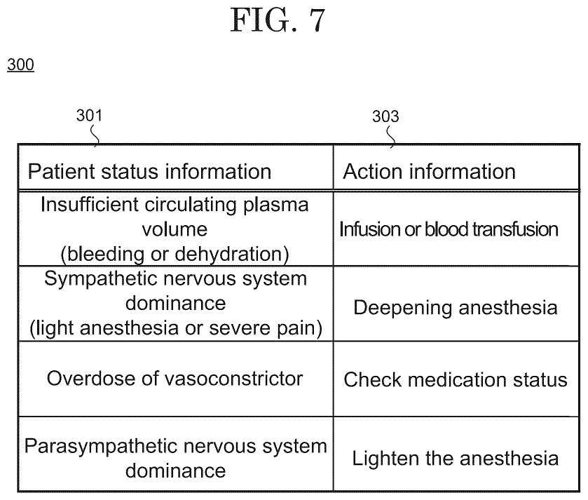

FIG. 7 is an action information data table 300. The action information data table 300 includes patient status information 301 and action information 303. The action information data table 300 indicates action information associated with the patient status information. In this example, the action information “infusion or blood transfusion” is associated with the patient status information “insufficient circulating plasma volume”.

### [1-5. Vital Data Evaluation Process]

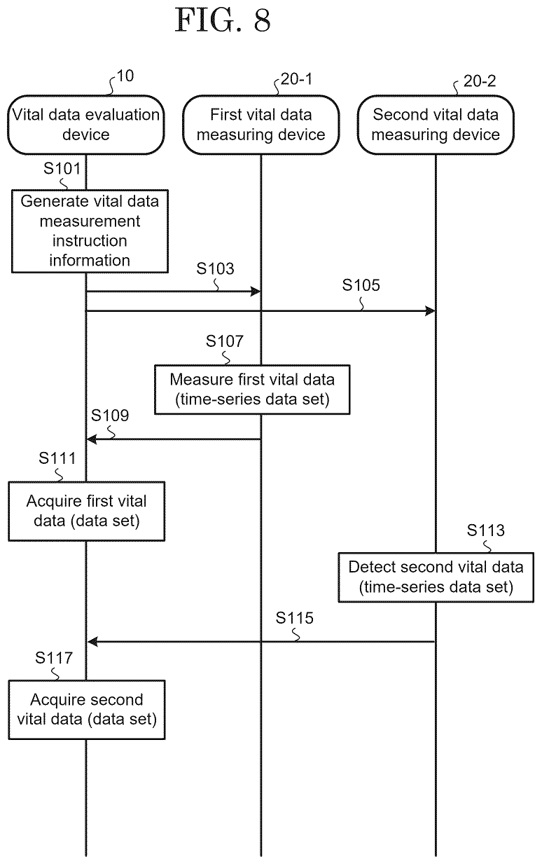

Next, the vital data evaluation process implemented in the vital data evaluation system according to the present embodiment will be described. FIG. 8 to FIG. 12 are flowcharts showing a vital data evaluation process of the vital data evaluation system 1 according to the present embodiment.

### [1-5-1. Acquisition of First Vital Data and Second Vital Data]

First, in FIG. 8, the vital data evaluation device 10 generates vital data measurement instruction information (step S101). For example, when a vital data measurement request is received from the terminal device 40 or another vital data measurement request is received, the vital data evaluation device 10 may generate the vital data measurement instruction information. The vital data evaluation device 10 transmits vital measurement instruction information to the first vital data measuring device 20-1 and the second vital data measuring device 20-2 (step S103 and step S105).

The vital data measurement instruction information may be set by an evaluator, such as a medical professional, or may be generated by the patient status information, patient information, environmental information, or the like, which will be described later (for example, the patient status information indicates “insufficient circulating plasma volume”, so the blood pressure measurement interval is shortened, or the like).

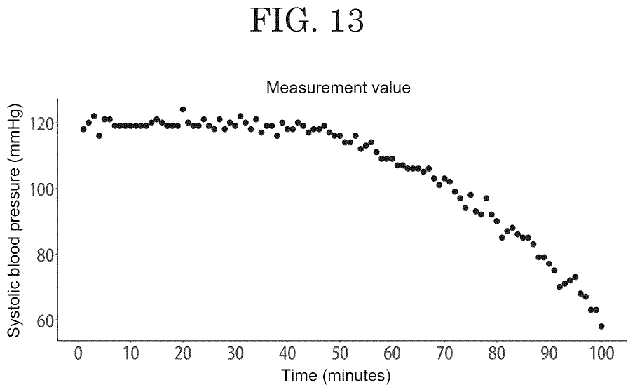

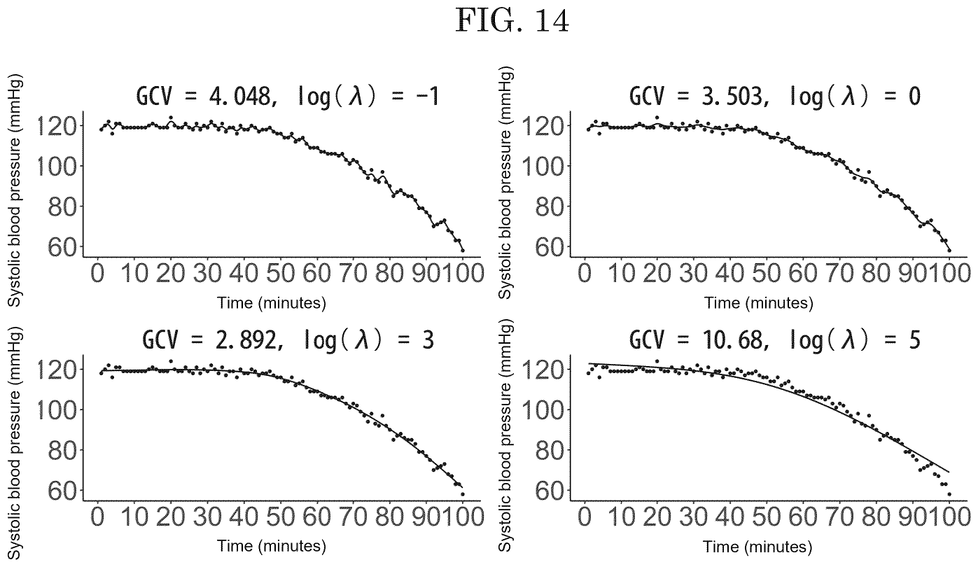

Based on the vital data measurement instruction information, the first vital data measuring device 20-1 measures one type of first vital data. In this example, the first vital measuring device measures systolic blood pressure (mmHg) (step S107). In this case, the first vital data measuring device may measure the first vital data for a predetermined period, or may continuously acquire the first vital data until a measurement end instruction is received. In addition, the measurement may be performed not based on the vital data measurement instruction information, but based on the instruction (information) directly input to the individual vital data measuring device. The measured first vital data is transmitted to the vital data evaluation device 10 as a data set arranged for each time series (step S109), and the vital data evaluation device 10 acquires the first vital data (step S111). The acquired first vital data (data set) is stored in the vital DB 50a. FIG. 13 is an example of the acquired first vital data set. As shown in FIG. 13, the acquired first vital data (data set) may be a discrete data set (scatter plot) in which time (minutes) is set on the horizontal axis and systolic blood pressure is set on the vertical axis. In this example, the vital data is data acquired every other minute.

Similarly, based on the vital data measurement instruction information, the second vital data measuring device 20-2 measures second vital data of a different type (second type) from the first vital data. In this example, the second vital data measuring device 20-2 measures the heart rate (bpm) (step S113). In this case, the second vital data measuring device 20-2 may measure the second vital data for a predetermined period, or may continuously acquire the second vital data until the measurement end instruction is received. In addition, the measurement may be performed not based on the vital data measurement instruction information, but based on the instruction (information) directly input to the individual vital data measuring device. The measured second vital data is transmitted to the vital data evaluation device 10 as a data set arranged for each time series (step S115), and the vital data evaluation device 10 acquires the second vital data (data set) (step S117). The acquired first vital data set and the acquired second vital data set correspond in time period.

Specifically, it means that the first vital data set and the second vital data set are present during a specific time period, such as from 10:00 to 11:00

### [1-5-2. Preprocessing of Vital Data]

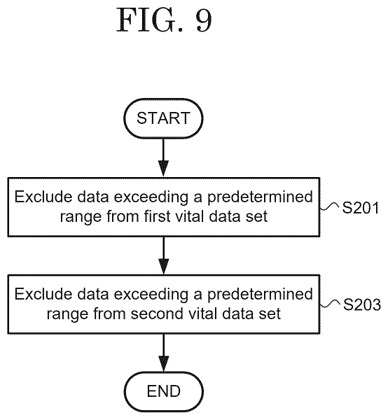

Next, as shown in FIG. 9, the vital data evaluation device 10 performs preprocessing of vital data. First, the vital data evaluation device 10 performs preprocessing of the acquired first vital data. In this example, data exceeding a preset numerical range is excluded (cleaned) from the first vital data set (step S201). Since the first vital data may be measured at a predetermined time interval, the first vital data set is discrete time-dependent data. When the function information is generated, if the first vital data set acquired in the function model is applied as it is, the function information is strongly affected by outliers. However, in the present embodiment, by performing the above-described preprocessing, the first vital data function information can be accurately generated, or the first vital data function information can be easily (or quickly) generated. The pre-processed first vital data set may be stored in the vital DB 50a. In this case, the first vital data set before preprocessing may be deleted from the vital DB 50a.

Next, the vital data evaluation device 10 performs preprocessing of the acquired second vital data. In the present embodiment, similar to the first vital data, the second vital data exceeding a preset range is excluded (cleaned) from the acquired second vital data set (step S203). The pre-processed second vital data set may be stored in the vital DB 50a. In this case, the second vital data set before preprocessing may be deleted from the vital DB 50a.

### [1-5-3. Generation of Function Information]

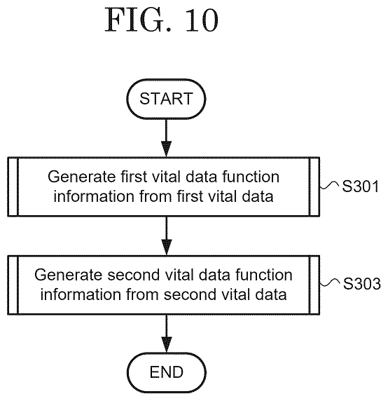

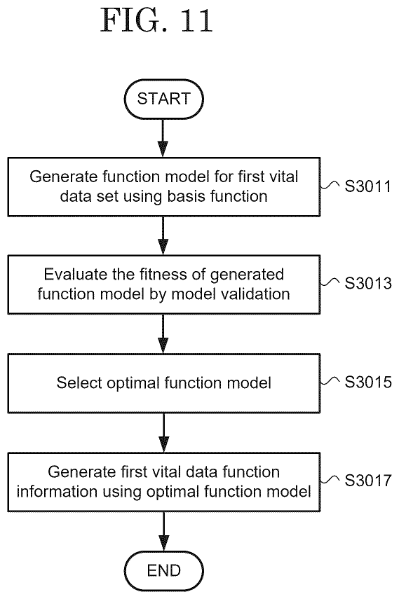

Next, as shown in FIG. 10, the vital data evaluation device 10 generates the vital data function information using the vital data set. First, the vital data evaluation device 10 generates the first vital data function information (step S301). FIG. 11 is a flowchart for generating first vital data function information.

In FIG. 11, first, the vital data evaluation device 10 generates a function model for the first vital data set (step S3011). When generating the function model, as shown in the data table of FIG. 4, a basis function to be used is selected from a plurality of basis functions prepared in advance. In this example, B-spline, Fourier, polygonal basis, polynomial basis, and constant basis are used as the basis function. B-spline has a feature of being easy to use. Fourier series includes sine and cosine functions, and has a feature of being easy to use when there is periodicity, such as diurnal variation. The polygonal basis is recommended when function regression is the goal. The polynomial basis has a feature that it fits well to the center of the data, but not to the tail. The constant basis has a feature that it fits well for data that does not change over time. Further, other basis functions may be used as the basis function. In addition, an autoregressive function, a moving average function, or the like that does not use a basis function may be used. The selection of the basis function or the function model may be made based on information associated with the target vital data, patient information, or external information (e.g., environmental information) associated with the patient. Further, in the present embodiment, a basis function or the like to be used may be set in advance depending on the type of vital signs. For example, in the present embodiment, as shown in FIG. 5, the B-spline is selected as a basis function for vital data of systolic blood pressure.

Next, the vital data evaluation device 10 analyzes and evaluates the fitness of the generated function model by model validation (step S3013). In this example, when a function model is generated by selecting B-spline as the basis function, validation may be performed using Generalized Cross Validation (GCV). An equation (Equation 1) used for GCV is shown below. In GCV evaluation, a function model is input into Equation 1. In this case, the smaller the GCV value, the higher the fitness of the function model.

$$GCV ( λ ) = \frac{{N}^{- 1} {\sum }_{n = 1}^{N} {( {Y}_{n} - {\hat{Y}}_{n} )}^{2}}{{( 1 - {N}^{- 1} trace ( {H}_{λ} ) )}^{2}} \qquad ( Equation 1 )$$

FIG. 14 is an example of the generated function model. In FIG. 14, the upper left is log(λ)=−1, the upper right is log(80 )=0, the lower left is log(λ)=3, and the lower right is log(λ)=5.

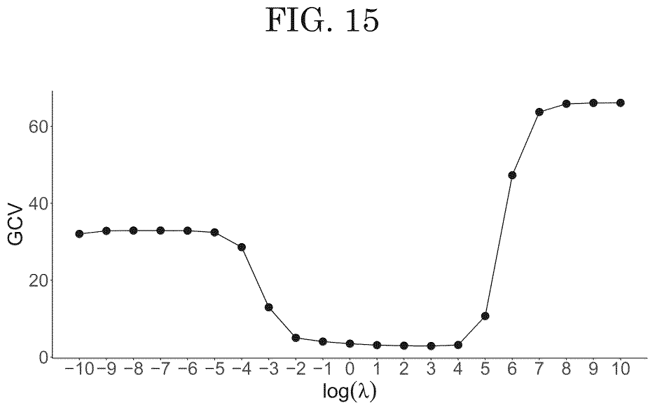

FIG. 15 is a graph of the horizontal axis log(λ), and the vertical axis GCV value for evaluating the function model. It can be seen from FIG. 15 that log(λ)=3 and the GCV is the smallest. As a result, a function model with log(λ)=3 is selected as the optimal function model using B-spline as the basis function (step S3015).

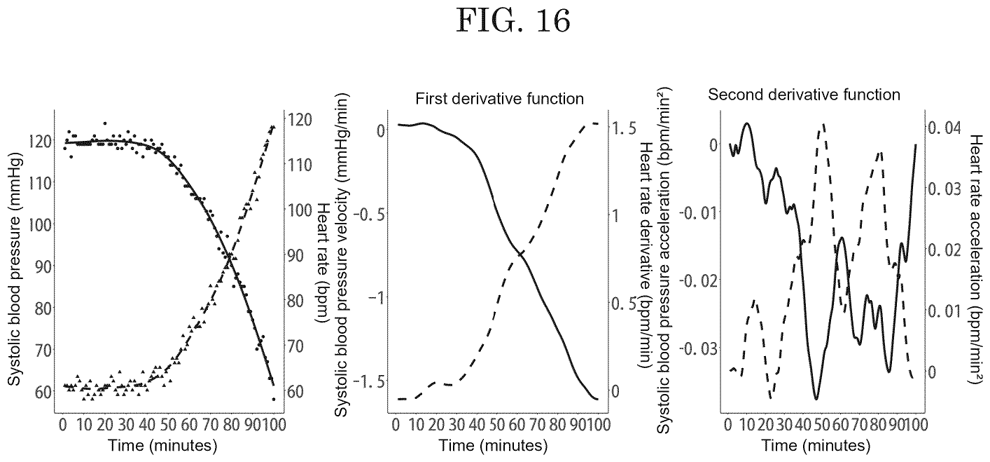

Finally, the vital data evaluation device 10 generates the first vital data function information for the first vital data set by fitting using the above-described optimal function model (in this example, log(λ)=3 is set using B-spline as the basis function) (step S3017) (see the left figure of FIG. 16: the circle is the first vital data set, the solid line is the first vital data function information, the triangle is the second vital data set, and the broken line is the second vital data function information).

Next, the vital data evaluation device 10 generates the second vital data function information for the second vital data set (step S303). Since the method for generating the second vital data function information is substantially the same as the first vital data function information, the description thereof will be omitted.

Further, in an embodiment of the present disclosure, the vital data evaluation device 10 may adjust the penalty term when evaluating the function model. The penalty term represents a constraint on the parameters of the function model and improves the generalization performance of the function model. The penalty term includes L1 regularization (Lasso regularization), L2 regularization (Ridge regularization), Elastic Net regularization, and the like. For example, in the case of vital data of variable vital signs (e.g., heart rate, etc.), the penalty term may be set to be weaker. The penalty term may be set to be weaker in situations where vital signs fluctuate significantly (e.g., in operating rooms). Further, when long-term trends are desired, the penalty term may be set to be stronger.

### [1-5-4. Analysis of Vital Data Function Information, Generation of Patient Status Information and Action Information]

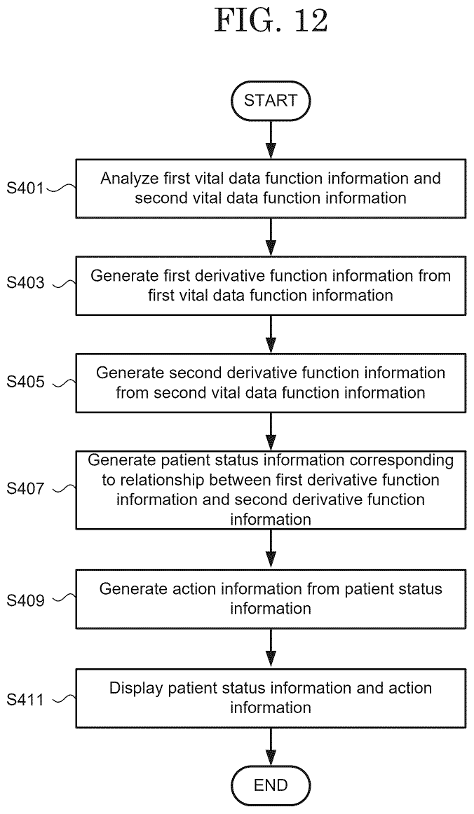

Next, in the flowchart of FIG. 12, the vital data evaluation device 10 analyzes the generated first vital data function information and the generated second vital data function information (step S401). In this case, the vital data evaluation device 10 generates the first vital data derivative function information based on the first vital data function information (step S403). In this example, the first vital data derivative function information is generated by performing a first-order differential calculation processing on the first vital data function information (see the central view of FIG. 16: the solid line is the first vital data derivative function information).

Similarly, the vital data evaluation device 10 generates the second vital data derivative function information based on the second vital data function information (step S405). In this example, the second vital data derivative function information is generated by performing the first-order differential calculation processing on the second vital data function information (see the central view of FIG. 16: the broken line is the second vital data derivative function information).

Further, in the present embodiment, the vital data evaluation device 10 generates the first identification information associated with the first vital data derivative function information. Similarly, the vital data evaluation device generates the second identification information associated with the second vital data derivative function information. The first identification information is information that allows the transition of the first vital data to be visually recognized. Specifically, as shown in FIG. 6, in the case where the first vital data (in this example, systolic blood pressure) is in an upward trend, the first identification information 201m is an arrow pointing to the upper right. In the case where the first vital data (systolic blood pressure) is in a downward trend, the first identification information 201m is an arrow pointing to the lower right. The second identification information is information that allows the transition of the second vital data to be visually recognized, and has an arrow shape similar to the first identification information. In addition, the shape (size, thickness, color, and the like) of the arrow may be changed according to the sign and magnitude of the derivative function, and the shape may not be an arrow as long as the direction can be determined. The first identification information and the second identification information may be displayed (output) on the display unit 14. This makes it easier for medical professionals to track the transition of the vital data.

Next, the vital data evaluation device 10 generates patient status information based on the relationship between the first vital data derivative function information and the second vital data derivative function information (step S407). The patient status information is information for estimating the patient's status. In this example, the patient status information is selected from the patient status data table 200 (also referred to as “patient status data set”) shown in FIG. 6. Specifically, when the first vital data function information f₁′ is negative (−) (decrease in systolic blood pressure) and the second vital data function information f₂′ is positive (+) (increase in heart rate), the vital data evaluation device 10 generates the patient status information “insufficient circulating plasma volume”. The vital data evaluation device 10 displays the generated patient status information on the display unit 14. This allows medical professionals to assess the patient's status.

Next, the vital data evaluation device 10 generates action information based on the patient status information (step S409). In this example, the action information is selected from the action information data table 300 (also referred to as “action information data set”) shown in FIG. 7. The action information is associated with the patient status information. In this example, the action information “infusion or blood transfusion” associated with the patient status information “insufficient circulating plasma volume” is generated. The vital data evaluation device 10 displays the generated correspondence data on the display unit 14 (step S411). This allows medical professionals to assess an action method for the patient. As described above, the vital data evaluation method according to the present embodiment ends.

According to the present embodiment, a function (vital data function information) is generated from two types of vital data measured for a predetermined period, thereby it is possible to accurately capture changes and transitions in vital data. In addition, based on the relationship between the functional information of the two types of vital data, the patient status information and the action information can be easily generated. It is also possible to respond to changes in circumstances by adding information such as vital data measured in real time. That is, by using the present embodiment, it is possible to easily determine the patient's status similar to that of a skilled medical professional.

In this case, an example of an actual medical procedure based on the action information obtained by the present embodiment will be described. The first vital data and the second vital data may be acquired after the infusion or blood transfusion treatment is started based on the action information “infusion or blood transfusion”. Next, the vital data evaluation device 10 generates the first vital data function information and the second vital data function information based on the acquired first vital data and the acquired second vital data. Further, the vital data evaluation device 10 generates the first vital data derivative function information and the second vital data derivative function information based on the first vital data function information and the second vital data function information. In this case, in the case where the “systolic blood pressure is in an upward trend” based on the first vital data derivative function information and the “heart rate is in a downward trend” based on the second vital data derivative function information, even if the vital data or the vital data function information fluctuates within the abnormal value range, the vital data evaluation device can analyze and determine that the symptom of the patient is being stabilized. Then, in the case where the actual systolic blood pressure and heart rate fall within the normal range, the vital data evaluation device can analyze and evaluate that the patient's status is stable.

Further, in an embodiment of the present disclosure, only the sign (positive and negative information) of the first derivative function information is used as the first vital data derivative function information and the second vital data derivative function information for simplification, but the numerical information may be used. In addition, although only the first derivative function information is used, the second derivative function information may be used in addition to the first derivative function information. These allow for a more detailed evaluation of the degree of change.

Further, in an embodiment of the present disclosure, although an example has been described in which the vital data evaluation device 10 displays the generated patient status information and action information on the display unit 14, the present disclosure is not limited to this. For example, the vital data evaluation device 10 may output the patient status information and/or the action information in an audio format or to an external device (the terminal device 40).

Further, in an embodiment of the present disclosure, although an example of generating the patient status information based on the first vital data derivative function information and the second vital data derivative function information has been described, the present disclosure is not limited to this. For example, in addition to the first vital data derivative function information and the second vital data derivative function information, the patient status information may be generated using the first vital data, the first vital data function information, the second vital data, the second vital function information, the patient information, or the environmental information. The patient information includes at least one piece of information, such as sex, age, occupation, and test value of the patient. The environmental information includes information related to the patient, such as time information, spatial information such as an operating room or hospital room, information related to the administration of medication, and the like. The patient information and the environment information may be information that changes with time. With respect to the information that changes with time, a function model may be generated, similar to the vital data, and function information and derivative function information may be generated. As a result, more accurate patient status information can be generated. The above-described information may also be used when the action information is generated.

### Second Embodiment

In the present embodiment, a patient status evaluation method different from the first embodiment will be described. Specifically, an example of generating the first vital data derivative function information and the second vital data derivative function information when the first vital data function information exceeds (or falls below) a threshold value will be described.

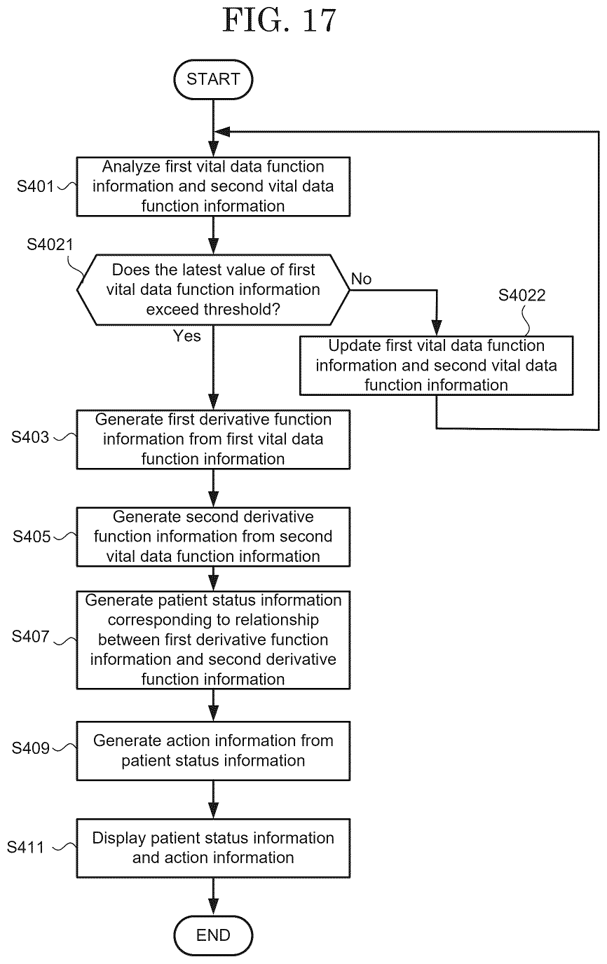

FIG. 17 is a flowchart of the vital data evaluation method. As shown in FIG. 17, when analyzing the relationship between the first vital data function information and the second vital data function information, whether the first vital data function information satisfies a predetermined condition may be determined (step S4021). In this example, the vital data evaluation device 10 determines whether the latest value (or the current value) in the function information of the “systolic blood pressure” is less than 100 (exceeds the threshold value). When the latest value in the function information of the “systolic blood pressure” is 100 or more (step S4021; No), a first vital data evaluation device updates the first vital data function information and the second vital data function information (step S4022), and returns to step S401. When the latest value in the functional information of the “systolic blood pressure” is less than 100 (step S4021; Yes), the first vital data derivative function information and the second vital data derivative function information may be generated. The subsequent processing is similar to that of the first embodiment. In addition, vital data may be used in the determination in S4021. In the present embodiment, since the calculation processing for generating vital data derivative function information is performed in response to changes in the patient's symptoms, it is possible to easily determine the patient's status similar to that of a skilled medical professional while suppressing the calculation load. Further, in the case where there is a margin in the calculation function, the vital data derivative function information may be calculated at all times.

In the present embodiment, when the first vital data or the first vital data function information f₁ falls below the cutoff value (or when the second vital data or the vital data function information f₂ exceeds the cutoff value), the above processing may be executed.

### Third Embodiment

In the present embodiment, an example including the severity when generating the patient status information will be described.

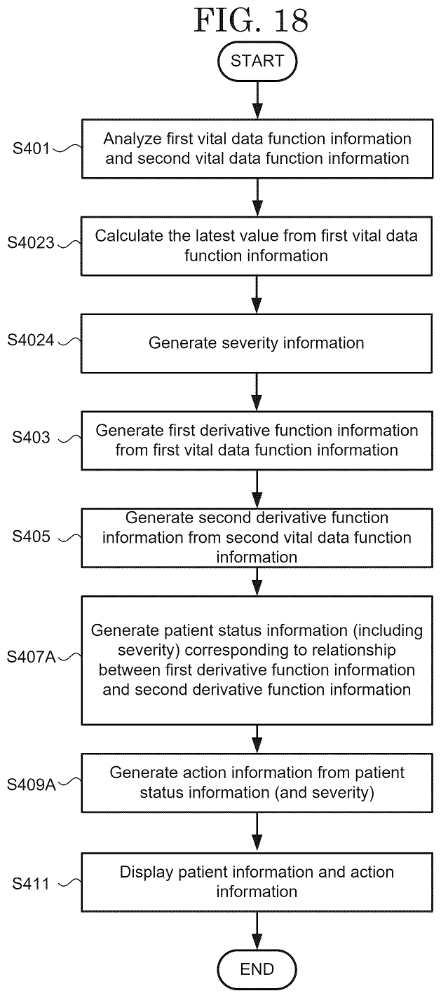

FIG. 18 is a flowchart of patient status information generation. As shown in FIG. 18, the vital data evaluation device 10 analyzes the first vital data function information (step S401). In this case, the vital data evaluation device 10 calculates the latest value (current value) of the first vital data (step S4023). The vital data evaluation device 10 may generate the severity information based on the latest information of the first vital data function information (step S4024). Specifically, the first vital data is determined as “low severity” when the “systolic blood pressure” is less than 100, as “high severity” when the “systolic blood pressure” is less than 80, and as “emergency situation” when the “systolic blood pressure” is less than 60. In the present embodiment, the vital data evaluation device 10 may add the severity to the patient status information generated in the first embodiment (step S407A). The term “severity” refers to the degree of mildness, severity, and urgency of the patient's status.

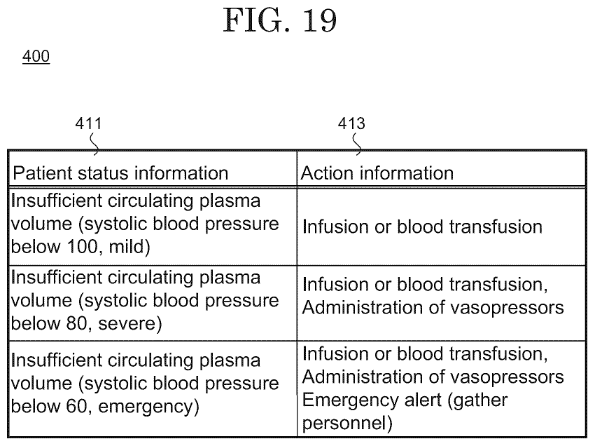

Further, the vital data evaluation device 10 generates action information based on the severity and the patient status information (step S409A). FIG. 19 is an example of an action information data table 400 according to the present embodiment. In this example, similar to the first embodiment, it is assumed that the first vital data function information f₁′ is negative (−) (decrease in systolic blood pressure) and the second vital data function information f₂′ is positive (+) (increase in heart rate). The action information data table 400 includes patient status information 411 and action information 413. As shown in FIG. 19, the patient status information 411 includes severity information in addition to the patient status information. The action information 413 is associated with the patient's status and severity. Specifically, in the case where the patient status information is “insufficient circulating plasma volume (systolic blood pressure 100 or less, low severity)”, the action information “infusion or blood transfusion” is set. In the case of “insufficient circulating plasma volume (systolic blood pressure 80 or less, high severity)”, the action information “administration of vasopressor” is set in addition to “infusion or blood transfusion”. Further, in the case of “insufficient circulating plasma volume (systolic blood pressure 60 or less, emergency situation)”, the action information “emergency alert (gather personnel)” is set in addition to “infusion or blood transfusion and administration of vasopressors”. Therefore, by using the present embodiment, it is possible to propose an action by dividing the patient's status into severity levels, and it is possible to easily determine the patient's status similar to that of a skilled medical professional.

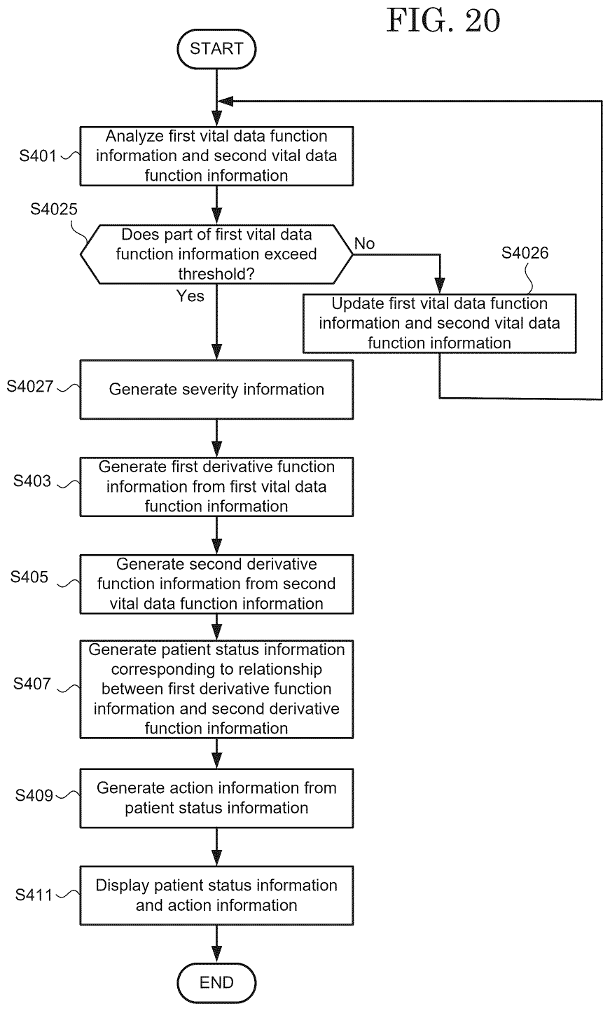

Further, in the present embodiment, the vital data evaluation device 10 may generate the level of severity based on a predetermined condition. For example, as shown in FIG. 20, the vital data evaluation device 10 determines whether the first vital data function information satisfies a predetermined condition (exceeds the threshold value) (step S4025). In this example, the vital data evaluation device 10 determines whether the latest value in the function information of the “systolic blood pressure” is less than 100. When the latest value of the function information of the “systolic blood pressure” is 100 or more (step S4025; No), the first vital data evaluation device updates the first vital data function information and the second vital data function information (step S4026), and returns to step S401. When the latest value of the functional information of the “systolic blood pressure” is less than 100 (step S4025; Yes), the vital data evaluation device 10 generates the severity information (step S4027). The severity information may be generated based on the latest value of the first vital data function information.

Further, although only positive and negative information of the derivative function information is used for simplification in the present embodiment, numerical information may be used. In addition, although only the first derivative function information is used, the second derivative function information may be used in addition to the first derivative function information. These allow for a detailed evaluation of severity reflecting the degree of change. Further, similar to the first embodiment, in addition to these pieces of information, the severity information may be generated using the first vital data, the first vital data function information, the second vital data, the second vital function information, the patient information, or the environmental information. In this case, more detailed severity information of the patient's status may be generated by combining the severity based on the first vital data (the first vital data function information and the first vital data derivative function) and the severity based on the second vital data (the second vital data function information and the second vital data derivative function).

### Fourth Embodiment

In the present embodiment, a method for generating vital data derivative function information different from that in the first embodiment will be described. Specifically, an example of generating and outputting a state transition marker as vital data derivative function information will be described.

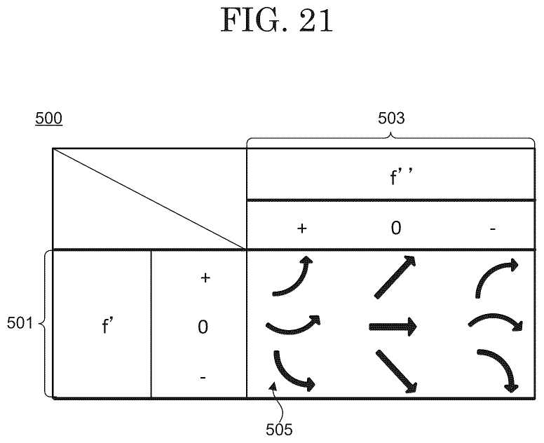

FIG. 21 is a state transition marker data table 500. The state transition marker data table 500 includes first-order derivative function information 501, second-order derivative function information 503, and a transition marker 505. In the present embodiment, the vital data evaluation device 10 generates (calculates) the first-order derivative function and the second-order derivative function from the vital data function information (see the central and right figures in FIG. 16). Based on the state transition marker data table 500 of FIG. 21, the vital data evaluation device 10 generates a transition display marker as vital data derivative function information from a combination of the generated first derivative function and second derivative function.

Specifically, the shape of the mark is determined using the value of the first-order derivative function f′ and the value of the second-order derivative function f″. For example, it is assumed that the vital data function information is in the state of Equation 2 at the 100-minute point (t=100). In this case, the equations and numerical values are simplified for the sake of explanation.

$$f ( t ) = 2 {t}^{2} - 19890 \qquad ( Equation 2 )$$

When the derivative (first and second) is obtained from this,

$${f}^{′} ( t ) = 4 t$$

$${f}^{″} ( t ) = 4$$

In this case, f, f′, and f at the time t=100 minutes (current value) are the following values.

$$f ( 10 \; 0 ) = 2 * {100}^{2} - 19890 = 110$$

$${f}^{′} ( 100 ) = 4 * 100 = 400$$

$${f}^{″} ( 100 ) = 4$$

As described above, when the first-order derivative function f′ is 400, that is, “+”, and the second-order derivative function f″ is 4, that is, “+”, the arrow points to the upper right and is convex downward non-linearly, indicating a rapid increase (upper left of the transition marker 505 in FIG. 21). In another example, when the first-order derivative function f′ is “+” and the second-order derivative function f″ is “0”, the transition marker is represented as a straight arrow pointing upward to the right. (upper center of the transition marker 505 in FIG. 21). This arrow represents that the vital data shows a steady increase. In another example, when the first-order derivative function f′ is “+” and the second-order derivative function f″ is “−”, the arrow takes the form of an upwardly convex arrow pointing to the upper right, indicating that the increase becomes more gradual (upper right of the transition marker 505 in FIG. 21). It is also possible to display the degree of change by reflecting the degree of “+” or “−” in the size or shape of the arrow. In addition, if the direction is known, the display format does not have to be an arrow. Further, the second-order derivative function may be omitted for the purpose of simplifying the display and calculation processing.

Further, in the present embodiment, the vital data evaluation device 10 can generate the patient status information and the action information based on a relationship between a first vital data transition marker generated based on the first vital data function information and a second vital data transition marker generated based on the second vital data function information.

By using the present embodiment, it is possible to grasp the transition of vital data in more detail. Therefore, the patient status information can be generated more accurately. Further, in the present embodiment, by displaying the generated transition display marker, the transition of the patient vital data (the transition of the patient's status) can be visually and quickly recognized in detail.

### Fifth Embodiment

In the present embodiment, an example of generating patient status information by machine learning patterns of the first vital data function information and the second vital data function information will be described. In addition, descriptions of parts that overlap the first embodiment of the present disclosure will be omitted as appropriate.

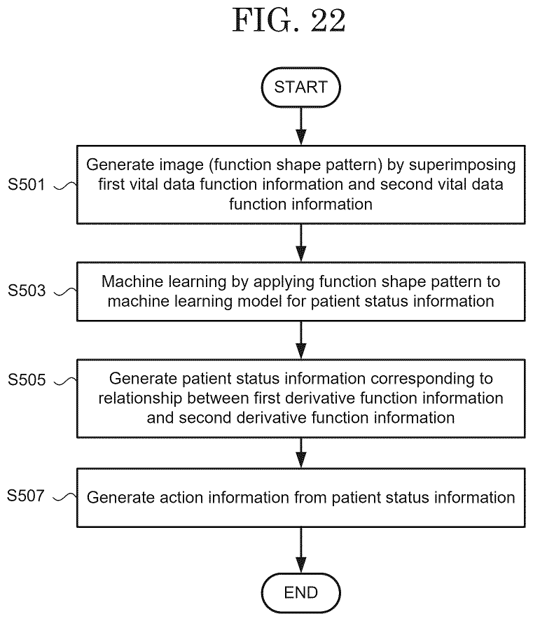

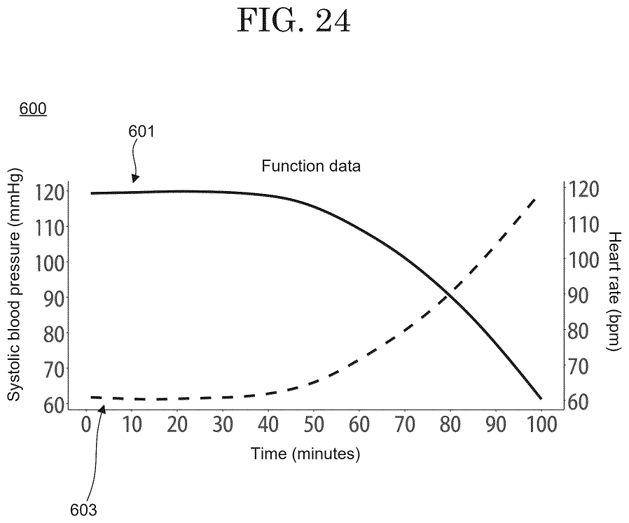

FIG. 22 is a flowchart showing vital data evaluation processing according to the present embodiment. First, the vital data evaluation device 10 generates an image in which the generated first vital data function information and the generated second vital data function information are superimposed (also referred to as a “function shape pattern”) (step S501). FIG. 24 is an example of the function shape pattern 600. As shown in FIG. 24, the function shape pattern 600 includes first vital data function information 601 and second vital data function information 603, but may also include the first vital data and the second vital data.

Next, the generated function shape pattern is applied to a machine learning model for generating the patient status information, and machine learning is performed (step S503). The machine learning model for generating the patient status information corresponds to the shape pattern of the first vital data function information and the second vital data function information.

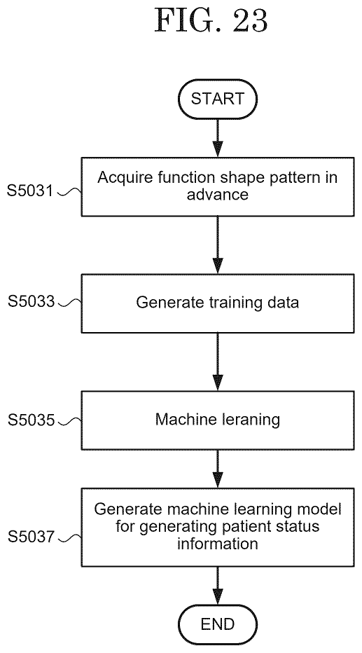

FIG. 23 is a flowchart of the machine learning model generation for generating patient status information according to the present embodiment. As shown in FIG. 23, the function shape pattern for generating the machine learning model for generating patient status information is acquired in advance (step S5031).

Next, training data is generated based on the function shape pattern acquired in advance (step S5033). In this case, the training data may be arbitrarily generated by an input from the user. Specifically, the training data may be generated by inputting information corresponding to the actual patient's status and the transition of the vital data. Further, the patient information or the environment information, as used in the first embodiment, may be used as the training data.

Next, machine learning is performed using the training data and the function shape pattern acquired in advance (step S5035). Known learning methods such as Backpropagation and Genetic Algorithm (GA) may be used for the machine learning. By repeatedly performing the machine learning, the machine learning model for generating the patient status information is generated (step S5037).

Returning to FIG. 22, the generated function shape pattern is applied to the machine learning model to perform machine learning, and as a result, the vital data evaluation device 10 generates (outputs) patient status information (step S505). Next, the vital data evaluation device 10 generates action information based on the generated patient status information (step S507). This completes the vital data evaluation method according to the present embodiment.

By using the present embodiment, it is possible to recognize the pattern of the function shape acquired from the two vital data by machine learning, and facilitate understanding of the patient's status. That is, by using the present embodiment, it is possible to easily determine the patient's status similar to that of a skilled medical professional.

Further, in the present embodiment, image information obtained by superimposing the first vital data function information and the second vital data function information or a marker indicating a change or a status as in the fourth embodiment may be displayed or output to the terminal device 40. As a result, the relationship between the first vital data function information and the second vital data function information can be visually recognized.

Further, in the present embodiment, when the machine learning is performed, the training data may be generated by appropriately combining at least one of the vital data and the derivative function information, and the machine learning may be performed. Further, in the present embodiment, although an example in which an image in which two pieces of vital data function information are physically superimposed is used has been described for clarity of explanation, the present disclosure is not limited to this. For example, if it is found that the time on the horizontal axis coincides with each other, an individual image may be used for each vital data function information. In addition, although an image is used for learning in the present example, the information used for learning is not limited to an image, and may be a function or numerical data that is the source of the image, or may be used together with an image. The function, the numerical data thereof, or the image data may be used as information for prediction or may be used as data to be predicted. Although the model with training is used in the present example, it may be a model without training. In addition, when the present embodiment is applied to an individual patient, the machine learning model may be optimized for the individual patient or for the user of the model by utilizing the difference between the predicted value of the patient status information and the actual patient status information. In this case, real-time feedback may be provided for relearning. As a result, a model corresponding to the user is constructed, so that the patient status information can be predicted more precisely.

### Modifications

The present disclosure is not limited to the above-described embodiments, and includes various other modifications. For example, the above-described embodiments have been described for the purpose of explaining the present disclosure in an easy-to-understand manner, and are not necessarily limited to those having all the described configurations. In addition, a part of a configuration of an embodiment may be replaced with a configuration of another embodiment, and a configuration of another embodiment may be added to a configuration of an embodiment (the configuration of each embodiment may be combined). Further, it is possible to add, delete, or replace a part of the configuration of each embodiment with another configuration. Some modifications will be described below. In addition, the examples in which the embodiments are modified can also be applied as examples in which the other embodiments are modified.

(1) An embodiment of the present disclosure can be applied not only to patients but also healthy individuals. In addition, an embodiment of the present disclosure can be applied not only to humans but also to animals. Further, it is possible to provide information, such as the subject's status, not only to medical professionals but also to the subject and non-medical professionals. The subject to which information is provided may be an Al or the like. Further, the vital data used in the present disclosure may be acquired not only from a medical device but also from a general device, a wearable terminal, or the like. In addition, the vital data and the system according to an embodiment of the present disclosure may be mounted not only on a medical device but also on a general device, a wearable terminal, or the like, or may cooperate with an external device.

(2) In the first embodiment of the present disclosure, although an example in which two types of vital data are measured and the patient's status is estimated from the relationship between two types of function information has been described, the present disclosure is not limited to this. For example, three or more types of vital data may be measured, and the patient's status may be estimated from a relationship between three or more pieces of functional information. Further, similar time-dependent data that is recorded continuously, like vital data, may also be treated similar to vital data. As a result, the patient's status can be grasped more accurately. Further, in a situation where there is less need to combine more than one type of vital data, the patient's status may be estimated from the functional information of one type of vital data.

(3) In an embodiment of the present disclosure, the value of the vital sign function information is used as the cutoff value, but the actually measured value may be used as the cutoff value. This function information and the actually measured value may be used together.

(4) In an embodiment of the present disclosure, the first vital data may be associated with vital data different from the second vital data when pre-processing the first vital data. For example, in the case where the “pulse pressure value (difference between the systolic blood pressure and the diastolic blood pressure)” which is the third vital data is less than the threshold value with respect to the “systolic blood pressure (value)” which is the first vital data, it is strongly suspected that there is an error in the measurement, so that the temporal corresponding systolic blood pressure value may be excluded. This makes it difficult to be affected by measurement errors, so that the vital data function information becomes accurate, thereby enabling a more accurate grasping of the transition of vital data.

(5) In an embodiment of the present disclosure, although it is assumed that the patient status information and the action information are displayed and medical professionals or the like respond according to the information, the present system may perform the response directly without the intervention of medical professionals or the like for events that can be handled without medical professionals or the like. In this case, the vital data evaluation system may include a medical device, an air conditioning device, and other electronic devices. For example, various processes such as adjusting the dose of medicine by a medical device and adjusting the room temperature by an air conditioning device may be executed based on the instruction information generated by the control unit of the vital data evaluation device.

(6) In the case where the same vital data is simultaneously measured by a plurality of devices, preprocessing of the vital data may be performed in consideration of measurement errors peculiar to the vital data. For example, when the second vital data “heart rate” is measured using two second vital data measuring devices, if the “heart rate” measured on the electrocardiogram is about twice the “heart rate” measured on the pulse oximeter, the “heart rate” on the electrocardiogram may be considered as a double count and excluded, or may be divided by two. This makes it less likely to be affected by measurement errors, and the vital data function information can therefore be easily generated. As a result, a more accurate grasp of vital data is enabled.

(7) In an embodiment of the present disclosure, when the first vital data is measured, the first vital data may be measured not only by one first vital data measuring device but also by a plurality of first vital data measuring devices. For example, blood pressure measurement using a cuff (manchette) is non-invasive and can be widely used in everyday life and hospitals, but shortening the measurement interval is limited. In addition, frequent repeated measurements may lead to skin damage at the measurement site. On the other hand, blood pressure measurement using an invasive arterial pressure measuring device can continuously monitor the blood pressure even though it is invasive, so that it is mainly used when the subject's blood pressure changes significantly. Therefore, the period during which invasive arterial pressure measurement is performed is often short. In addition, when blood pressure is measured using the invasive arterial pressure measuring device, blood pressure measurement using a cuff is often discontinued or performed less frequently. Therefore, when data measured by one of the first vital data measuring devices is used, a missing value may be generated in a certain period. By using the data measured by the plurality of first vital data measuring devices together, when the measurement value of the first vital data is interrupted in one first vital data measuring device (the first vital data is missing for a predetermined period), it is possible to supplement the first vital data, and an uninterrupted (stable) time-series data set for a predetermined period can be generated.

(8) Measuring the first vital data using the plurality of first vital data measuring devices makes it robust against measurement errors. For example, measurement of “heart rate” using the electrocardiogram is vulnerable to electrocautery, electromyography, and vibration. On the other hand, measurement of heart rate (pulse rate) using a pulse oximeter is vulnerable to blockage of blood flow, obstacles at the measurement site (nail polish, etc.), and movement of the limbs. Even if the plurality of first vital data measuring devices is used as described above, and if the measurement result can be considered to be medically almost identical by any first vital data measuring device, it can be expected that generating the first vital data function from both measurement values will make the measurement robust against measurement errors.

(9) In one embodiment of the present disclosure, although an example is shown in which the first vital data derivative function information and the second vital data derivative function information are used when generating the patient status information, the action information, and the transition marker based on the relationship between the first vital data function information and the second vital data function information, the present disclosure is not limited to this. For example, the patient status information, the action information, and the transition marker may be generated from the relationship between the integral value of the first vital data function information and the integral value of the second vital data function information.

For example, it is determined that the patient is in a state of (mild) hypotension based on the time integral value or the like of the numerical values below the threshold value in the first vital data (blood pressure) function, and it is determined that the patient is in a state of (mild) tachycardia based on the time integral value or the like of the numerical values above the threshold value in the second vital data function information (heart rate). Further, the patient status information “insufficient circulating plasma volume” and the action information “infusion or blood transfusion” can be generated from the combination of the integrated value of the first vital data function and the integrated value of the second vital data function information. In addition, a transition marker may be generated so as to indicate that the blood pressure transitions at a low level and the heart rate transitions at a high level. In this case, an analysis of whether there is an abnormality may be performed, or the severity information may be generated based on the integrated value of the first vital data function and the integrated value of the second vital data function information. Further, the parameters of the cutoff value may be changed from the integral value (e.g., if an outlier persists, increasing the sensitivity to derivative function information, etc.).

(10) In an embodiment of the present disclosure, although an example is shown in which the vital data derivative function information (the velocity of the vital data function information in the case of the first derivative and the acceleration of the vital data function information in the case of the second derivative) is evaluated and analyzed from the vital data function, the patient status information may be generated from the relationship with the vital data function information or the variance value of the vital data derivative function information. For example, it can be determined that the patient's status is becoming unstable based on a large velocity variance value.

(11) In an embodiment of the present disclosure, prediction information regarding changes in the first vital data can be generated by performing calculation processing on the first vital data functional information. For example, a short-term moving average (short-term line) and a long-term moving average (long-term line) of the first vital data can be prepared, and changes of the first vital data can be predicted from the relationship between the long-term line and the short-term line. More specifically, when the short-term line exceeds the long-term line, the vital data evaluation device 10 may use the moving average as information for generating the patient status information or a transition marker indicating that the first vital data is currently increasing. In addition, prediction information indicating that the first vital data will increase in the future may be generated. Other methods, such as calculation of a derivative function or the like by smoothing by methods not used in this example, changes directly calculated from the measured values (comparison with the measurement value from one hour ago, comparison with the previous measurement value, comparison with the average or median value over the last 10 minutes, etc.), regression (linear regression, etc.) of the measurement value over the last 15 minutes, correlation coefficient, and the like, can be used, and the patient status information and transition markers can be generated similarly. Further, information on changes may be used to determine the patient's status using not only the present time but also past changes or by combining information on past and current changes (e.g., if blood pressure has increased sharply in the past [a positive slope] and then leveled off at a high level [a zero slope], it is possible to determine that a treatment for lowering blood pressure is required, or to determine that there is a high risk because rapid increases and decreases are repeatedly occurring within a short period of time.).

(12) In an embodiment of the present disclosure, analysis processing may be performed on the generated patient status information and the action information based on the information input to the terminal device 40. For example, in the case where correction is required for the patient status information and the action information, the patient status information DB 50c and the action information DB 50d may be appropriately corrected based on the information input to the terminal device 40.

(13) In an embodiment of the present disclosure, the vital data evaluation device 10 may generate warning information when the first vital data function information (or the first vital data derivative function information) or the second vital data function information (or the second vital data derivative function information) satisfies a predetermined condition. Examples of the predetermined condition include the case where a part of the first vital data function information (for example, a maximum value or a minimum value) exceeds a preset numerical range, or the case where an integral value of the first vital data function information for a certain period exceeds a preset threshold. Further, when it is difficult to accurately calculate the integrated value, an approximate value of the integrated value may be calculated from the vital data itself by numerical integration or the like. In addition, the patient status information may be generated based on the relationship between the integral value of the first vital data function information and the integral value of the second vital data function information.

(14) Further, in an embodiment of the present disclosure, the measurement of the first vital data and the second vital data may be performed by a single measuring device.

(15) In the third embodiment of the present disclosure, although an example in which the vital data evaluation device 10 determines whether the first vital data function information satisfies a predetermined condition (exceeds a threshold value), the present disclosure is not limited to this. The vital data evaluation device 10 may determine whether the vital data (current value) satisfies a predetermined condition and then execute the next processing. As a result, it is possible to generate the severity information, the patient status information, or the action information using the data that can be normally assessed by medical professionals. In other embodiments, vital data evaluation processing may be performed using vital data as appropriate.

What is claimed is:

1. A vital data evaluation method comprising:
acquiring a first vital data set corresponding to a first type of vital data of a patient;
acquiring a second vital data set corresponding to a second type of vital data different from the first type of vital data of the patient;
generating first vital data function information based on the first vital data set;
generating second vital data function information based on the second vital data set; and
generating patient status information for estimating a patient's status based on a relationship between the first vital data function information and the second vital data function information.

2. The vital data evaluation method according to claim 1, further comprising:
excluding first vital data that exceeds a preset numerical range from the first vital data set;
excluding second vital data that exceeds a preset numerical range from the second vital data set;
generating the first vital data function information based on the first vital data set from which the first vital data exceeding the preset numerical range has been excluded; and
generating the second vital data function information based on the second vital data set from which the second vital data exceeding the preset numerical range has been excluded.

3. The vital data evaluation method according to claim 1, further comprising:
generating first vital data derivative function information based on the first vital data function information;
generating second vital data derivative function information based on the second vital data function information; and
generating patient status information based on a patient status data set corresponding to the first vital data derivative function information, the second vital data derivative function information, and a relationship therebetween.

4. The vital data evaluation method according to claim 3, wherein
the first and second vital data derivative function information are generated when at least one of the first and second vital data function information satisfies a predetermined condition.

5. The vital data evaluation method according to claim 1, further comprising:
generating action information for treating or managing the patient, based on an action information data set associated with the patient status information.

6. The vital data evaluation method according to claim 5, further comprising:
generating severity information based on at least one of the first and second vital data function information; and
generating the action information based on the severity information and the patient status information.

7. The vital data evaluation method according to claim 1, further comprising:
generating warning information when at least one of the first and second vital data function information satisfies a predetermined condition.

8. The vital data evaluation method according to claim 3, further comprising:
outputting first identification information associated with the first vital data derivative function information and second identification information associated with the second vital data derivative function information.

9. The vital data evaluation method according to claim 8, wherein
the first identification information is information that visually represents a transition of the first vital data; and
the second identification information is information that visually represents a transition of the second vital data.

10. The vital data evaluation method according to claim 1, further comprising:
selecting a basis function to be applied from a plurality of basis functions, based on at least one type of vital data, information about the patient, and external information associated with the patient, when generating at least one of the first and second vital data function information.

11. The vital data evaluation method according to claim 1, further comprising:
generating the patient status information by applying the first and second vital data function information to a pre-generated machine learning model.

12. The vital data evaluation method according to claim 11, wherein
the machine learning model corresponds to shape patterns of the first and second vital data function information.

13. A non-transitory computer-readable medium storing a program that causes a computer to execute the vital data evaluation method according to claim 1.

14. A vital data evaluation device comprising:
a processor; and
a memory device configured to store a program that causes the processor to:
acquire a first vital data set corresponding to a first type of vital data of a patient;
acquire a second vital data set corresponding to a second type of vital data different from the first type of vital data of the patient;
generate first vital data function information based on the first vital data set;
generate second vital data function information based on the second vital data set; and
generate patient status information for estimating a patient's status based on a relationship between the first vital data function information and the second vital data function information.

15. The vital data evaluation device according to claim 14, wherein the program causes the processor to:
exclude first vital data that exceeds a preset numerical range from the first vital data set;
exclude second vital data that exceeds a preset numerical range from the second vital data set;
generate the first vital data function information based on the first vital data set from which the first vital data exceeding the preset numerical range has been excluded; and
generate the second vital data function information based on the second vital data set from which the second vital data exceeding the preset numerical range has been excluded.

16. The vital data evaluation device according to claim 14, wherein the program causes the processor to:
generate first vital data derivative function information based on the first vital data function information;
generate second vital data derivative function information based on the second vital data function information; and
generate the patient status information based on a patient status data set corresponding to the first vital data derivative function information, the second vital data derivative function information, and a relationship therebetween.

17. The vital data evaluation device according to claim 16, wherein the program causes the processor to:
generate the first and second vital data derivative function information when at least one of the first and second vital data function information satisfies a predetermined condition.

18. The vital data evaluation device according to claim 14, wherein the program causes the processor to:
generate action information for treating or managing the patient, based on an action information data set associated with the patient status information.

19. The vital data evaluation device according to claim 18, wherein the program causes the processor to:
generate severity information based on at least one of the first and second vital data function information; and
generate the action information based on the severity information and the patient status information.

20. The vital data evaluation device according to claim 14, wherein the program causes the processor to:
generate warning information when at least one of the first and second vital data function information satisfies a predetermined condition.

21. The vital data evaluation device according to claim 16, wherein the program causes the processor to:
output first identification information associated with the first vital data derivative function information and second identification information associated with the second vital data derivative function information.

22. The vital data evaluation device according to claim 21, wherein
the first identification information is information that visually represents a transition of the first vital data; and
the second identification information is information that visually represents a transition of the second vital data.

23. The vital data evaluation device according to claim 16, wherein the program causes the processor to:
select a basis function to be applied from a plurality of basis functions, based on at least one type of vital data, information about the patient, and external information associated with the patient, when generating at least one of the first and second vital data function information.

24. The vital data evaluation device according to claim 14, wherein the program causes the processor to:
generate the patient status information by applying the first and second vital data function information to a pre-generated machine learning model.

25. The vital data evaluation device according to claim 24, wherein the machine learning model corresponds to shape patterns of the first and second vital data function information.

26. A vital data evaluation system comprising:
a processor; and
a memory device configured to store a program that causes the processor to:
acquire a first vital data set corresponding to a first type of vital data of a patient;
acquire a second vital data set corresponding to a second type of vital data different from the first type of vital data of the patient;
generate first vital data function information based on the first vital data set;
generate second vital data function information based on the second vital data set; and
generate patient status information for estimating a patient's status based on a relationship between the first vital data function information and the second vital data function information.

27. The vital data evaluation system according to claim 26, wherein the program causes the processor to:
exclude first vital data that exceeds a preset numerical range from the first vital data set;
exclude second vital data that exceeds a preset numerical range from the second vital data set;
generate the first vital data function information based on the first vital data set from which the first vital data exceeding the preset numerical range has been excluded; and
generate the second vital data function information based on the second vital data set from which the second vital data exceeding the preset numerical range has been excluded.
